(19) United States
(12) Patent Application Publication
(10) Pub. No.: US 2005/0261330 A1
(43) Pub. Date: Nov. 24, 2005

(54) Derivatives of 2,2,4-trisubstituted tetrahydrofuran an antifungal agents

(76) Inventors: Mohammad Salman, Haryana (IN); Ashwani Kumar Verma, Delhi (IN); Sanjay Malhotra, Delhi (IN); Ashok Rattan, Delhi (IN)

Correspondence Address: RANBAXY INC., 600 COLLEGE ROAD EAST, SUITE 2100, PRINCETON, NJ, 08540, US

(21) Appl. No.: 10/511,049

(22) PCT Filed: Apr. 12, 2002
(86) PCT No.: PCT/IB02/01197

§ 371 Date: Jul. 8, 2005

(51) Int. Cl. A61K 31/4745; A61K 31/4196; A61K 31/427; C07D 471/04

(52) U.S. Cl. 514/292; 514/381; 514/383; 546/81; 548/253; 548/263.4; 514/370

(57) ABSTRACT

The present invention relates to derivatives of 2,2,4-trisubstituted tetrahydrofuran as potential antifungal agents. This invention also relates to pharmaceutical compositions containing the compounds of the present invention and their use in treating and/or preventing the fungal infections in mammals, preferably humans.

### FIELD OF THE INVENTION

The present invention relates to derivatives of 2,2,4-trisubstituted tetrahydrofuran as potential antifungal agents.

This invention also relates to pharmaceutical compositions containing the compounds of the present invention and their use in treating and/or preventing the fungal infections in mammals, preferably humans.

### BACKGROUND OF THE INVENTION

Life-threatening, systemic fungal infections continue to be a significant problem in health care. In particular, patients who become “immunocompromised” as a result of diabetes, cancer, prolonged steroid therapy, organ transplantation anti-rejection therapy, the acquired immune deficiency syndrome (AIDS) or other physiologically or immunologically compromising syndromes, are especially susceptible to opportunistic fungal infections.

Since the 1950's and until recently, the key opportunistic fungal pathogens were Candida albicans, Aspergillus fumigatus and Zygomycetes, which cause mucormycosis, a rapidly fatal infection especially in diabetic patients. Today, non-albicans Candida isolates have become more frequent, as have other Aspergillus species. Candida species are now the fourth most common cause of nosocomial blood stream infection and they are associated with an extremely high mortality rate of 40%. From 1980 to 1990, the incidence of fungal infections in the US hospitals nearly doubled, from approximately 2 to 3.85 per 1000 patient days. The most marked increase in fungal infection rates occurred not only in transplant units or oncology centres, but also in surgical services. These changing patterns demonstrate that fungal infections are no longer limited to the most severely immunocompromised patients.

During the past two decades, a substantial shift in the epidemiology of candidemia due to different Candida species has occurred. In the 1960's and 1970's Candida albicans accounted for 85-90% of candidemia. In 1999 however, only 42% of candidemia cases were caused by C. albicans, while non-albicans Candida accounted for the remainder.

Cryptococosis is a leading cause of morbidity among the AIDS patients. The incidence of life threatening cryptococcal infection among these patients have been estimated to vary from 10 to 30%; 10-20% of the patients die during initial therapy and 30 to 60% patients succumb within a year. Penicillinium marneffei has been frequently isolated from HIV positive patients, especially in Southeast Asia.

The most common causative agent of mucormycosis is Rhizopus, a common bread mould that lives on any organic material. Other pathogens include Mucor, Rhizomucor and Absidia. Zygomycetes include twenty different fungi, all appearing the same histologically. The severely immunocompromised patient may become infected with Zygomycetes via respiratory inhalation.

Fusarium is the most prevalent plant fungus worldwide, and it is now recognized as a human pathogen as well. Fusarium infections can occur in immunocompetent or immunosuppressed individuals. Fusarium infection is life threatening and associated with a poor prognosis.

Penicillium marneffei is an environmental fungi that can cause serious, life threatening infections in immunosuppressed patients. Penicillium marneffei has gained particular attention during the AIDS pandemic, as it may produce disease that is clinically indistinguishable from disseminated histoplasmosis.

Invasive aspergillosis has become a leading cause of death, mainly among patients suffering from acute leukaemia or after allogenic bone marrow transplant and after cytotoxic treatment of these conditions. It also occurs in patients with condition such as AIDS and chronic granulomatous disease. At present, only Amphotericin B and itraconazole are available for treatment of aspergillosis. In spite of their activity in vitro, the effect of these drugs in vivo against Aspergillus fumigatus remains low and as a consequence mortality from invasive aspergillosis remains high.

Although the first agent with antifungal activity, Griseofulvin was isolated in 1939 and the first azole and polyene antifungal agents were reported in 1944 and 1949, respectively (Clin. Microbiol. Rev., 1988; 1:187), it was not until 1960 that Amphotericin B (I. J. Am. Acad, Dermatol, 1994; 31:S51), which is still the “gold standard” for the treatment of severe systemic mycoses, was introduced (Antimicrob. Agents Chemother. 1996, 40:279)). Despite the general effectiveness of Amphotericin B, it is associated with a number of complications and unique toxicities that limit its use. Furthermore, the drug is poorly absorbed from the gastrointestinal tract necessitating intravenous administration and also penetrates poorly into the cerebrospinal fluid (CSF) of both normal and inflamed meninges. The problems associated with Amphotericin B stimulated search for newer agents.

By 1980, members of the four major classes of antifungal agents, viz polyenes, azoles, morpholines and allylamines had been identified. And advances made during the 1990's led to the addition of some new classes such as the Candins, and the Nikkomycins (Exp. Opin. Investig. Drugs, 1997; 6:129). However, with 15 different marketed drugs worldwide, (Drugs, 1997; 53:539) the azoles are currently the most widely used and studied class of antifungal agents.

Azole antifungal agents prevent the synthesis of ergosterol, a major component of fungal plasma membranes, by inhibiting the cytochrome P-450 dependent enzyme lanosterol demethylase (referred to as 14-α-sterol demethylase or P-450 _{DM}). This enzyme also plays an important role in the cholesterol synthesis in mammals. When azoles are present in therapeutic concentrations, their antifungal efficacy is attributed to their greater affinity for fungal P-450_{DM} than for the mammalian enzyme (Curr. Opin. Chem. Biol., 1997; 1:176).

The azole antifungals currently in clinical use contain either two or three nitrogens in the azole ring and are thereby classified as imidazoles (e.g. ketoconazole, miconazole and clotrimazole) or triazoles (e.g. itraconazole and fluconazole), respectively. With the exception of Ketoconazole, use of the imidazoles is limited to the treatment of superficial mycoses, whereas the triazoles have a broad range of applications in the treatment of both superficial and systemic fungal infections. Another advantage of the triazoles is their greater affinity for fungal rather than mammalian cytochrome P-450 enzymes.

The use of Ketoconazole is severely restricted partly due to its poor toxicity and pharmacokinetic profile and also the fact that none of the opportunistic fungal infections like aspergillosis, candidemia and cryptococcosis are responsive to it (Antifungal Agents, pgs 401-410 In. G. L. Mandel, J. E. Benneft and R. Dolin (ed.) Principles and practice of infectious diseases, 4^{th} ed. Churchill Livingstone, Inc. New York, N.Y). Fluconazole is the current drug of choice for treatment of infections caused by Candida species and C. neoformans. However, management of serious infectious due to Candida species, are becoming increasingly problematic because of rising incidence of non-albicans species and the emergence non-albicans isolates resistant to both amphotericin B and the newer azoles. (Am. J. Med., 1996; 100:617). Also, fluconazole's spectrum suffers because it has only weak inhibitory activity against isolates of Aspergillus species. With regard to the prevention of invasive aspergillosis, a number of antifungal regimens have been suggested for neutropenic patients but only itraconazole has been considered for primary prophylaxis. However, its activity in the clinic remains mixed as it shows variable oral availability, low solubility and very high protein binding besides causing ovarian cancer in animals.

Voriconazole, the fluconazole analog launched recently by Pfizer exhibits 1.6 and 160 fold greater inhibition of ergosterol P450_{DM} in C. albicans and A. fumigatus lysates, respectively, compared to fluconazole (Clin. Microbiol. Rev., 1999; 12:40). The drawbacks associated with voriconazole are its non-linear pharmacokinetic profile besides some concern regarding its ocular toxicity. The development of some of the earlier compounds which included SCH 39304 (Genoconazole), TAK-187, SCH-42427 (Saperconazole), BAY R-8783 (Electrazole) and D-0870 had to be discontinued as a result of safety concerns.

ER-30346 (Ravuconazole), the fluconazole analog under development shows anti-aspergillus profile, at best only equal to that of itraconazole. Schering Plough's compound SCH 56592 (Posaconazole) shows potent broad spectrum activity against primary opportunistic fungal pathogens including Candida spp., C. neoformans and Aspergillus spp. However, it has a pharmacokinetic profile similar to that of itraconazole and is not detectable in CSF, even when the serum drug concentration after several days of treatment are 25 to 100 times above the MIC for the most resistant C. neoformans. (Antimicrobial Agents and Chemother, 1996; 40:1910, 36^{th} interscience Conference on Antimicrobial agents and chemotherapy, September 1996, New Orleans Abst. Drugs of the Future, 1996; 21:20).

The limited spectrum of antifungal activity, toxicity and lack of both an intravenous and an oral formulation for the same drug limit the likelihood of a successful patient outcome with available therapies.

Voriconazole was designed to retain the parenteral and oral formulation advantage of fluconazole while extending its spectrum to moulds, insufficiently treated yeasts and less common fungal pathogens. But though oral bioavailability of voriconazole is high, there is saturable metabolism which results in a more than proportional increase in exposure with increased oral and IV doses. Inter-individual variability in voriconazole pharmacokinetics is high and concerns about its occular toxicity potentials remain to be resolved.

Caspofungin is the first member of a new class of antifungal drugs (echinocandins). It reduces the synthesis of β(1,3)D-glucan, an essential structural cell wall component of fungi. The cell wall is a component of fungal cells that is not found in mammalian cells and loss of cell wall glucan results in osmotic fragility of the fungal organism. The activity of the drug on the cell wall is accomplished indirectly by non competitive inhibition of a gene whose product is a cell membrane protein responsible for glucan synthesis. But caspofungin is not active against Cryptococcus neoformans and is available only for IV use.

Thus, the antifungals in the market, as well as under development suffer with drawbacks such as toxicity, narrow spectrum of activity and fungistatic profile rather than fungicidal. Some of them also exhibit drug-drug interactions and as a result, therapy becomes complex. In view of the high incidence of fungal infections in immunocompromised patients and the recent trends for the steady increase of the population of such patients, demands for new antifungal agents with broad spectrum of activity and good pharmacokinetic properties has increased.

### SUMMARY OF THE INVENTION

The object of the present invention is to provide compounds of Formula I, 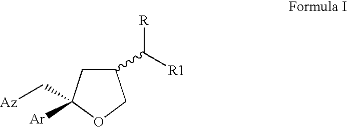
and its pharmaceutically acceptable salts, enantiomers, diastereomers, N-oxides, prodrugs, metabolites, polymorphs, or pharmaceutically acceptable solvates and pharmaceutical compositions containing these compounds which have anti fungal activity and overcome the problems associated with the azole compounds described in the prior art.

Accordingly, the present invention provides derivatives of 2,2,4-trisubstituted tetrahydrofuran compounds of Formula I.
wherein:
Az is a five to seven membered heterocyclic ring having one to four heteroatoms selected from N, S, or O; the preferred heterocyclic ring is 1,2,4-triazol-1-yl;
Ar is a five to seven membered heterocyclic ring containing one to four heteroatoms selected from the group consisting of oxygen nitrogen and sulphur; phenyl or a substituted phenyl group having one to three substituents independently selected from halogen (e.g. chlorine, fluorine, bromine or iodine), nitro, cyano, lower (C₁-C₄) alkyl, lower (C₁-C₄) alkoxy or perhalo lower (C₁-C₄) alkyl, perhalo lower (C₁-C₄) alkoxy; the preferred heterocyclic rings are thienyl and pyridyl;
R is H or methyl;
R₁ is selected from the group consisting of 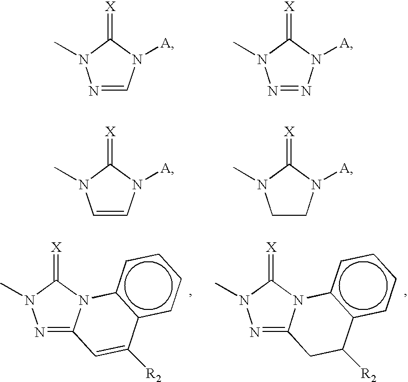
wherein
X is selected from the group consisting of CH₂, O, S and SO₂;
R₂ is hydrogen or lower(C₁-C₄) alkyl;
A is hydrogen, lower (C₁-C₄) alkyl, phenyl or phenyl substituted by one or more of groups independently selected from halogen (e.g. chlorine, fluorine, bromine or iodine atom), nitro, cyano, hydroxy, lower(C₁-C₄) alkyl, lower(C₁-C₄) alkoxy, perhalo lower(C₁-C₄)alkoxy or perhalo lower (C₁-C₄) alkyl, substituted or unsubstituted five or six membered heterocyclyl ring system containing one to four hetero atoms chosen from N, O and S, said heterocyclyl substituents being (C₁-C₈) alkanoyl, lower (C₁-C₄) alkyl, lower (C₁-C₄) alkoxy carbonyl, N,N-di(lower alkyl) (C₁-C₄) amino carbonyl, aminothiocarbonyl, N-lower(C₁-C₄) alkyl aminothiocarbonyl, N,N-di(lower alkyl) (C₁-C₄) aminothiocarbonyl, lower (C₁-C₄) alkyl sulfonyl, phenyl substituted lower (C₁-C₄) alkyl sulfonyl, N-lower(C₁-C₄) alkylamino, N,N-di(lower alkyl) (C₁-C₄) amino, 1,3-imidazol-1-yl, 2-loweralkyl (C₁-C₄) sulfenyl-1,3-imidazol-1-yl, pyridinyl, thiazolyl, 1,2,4 triazol-4-yl or phenyl or phenyl substituted by one or more of groups independently selected from halogen (chlorine, fluorine, bromine or iodine), perhalo lower(C₁-C₄) alkyl, perhalo lower(C₁-C₄) alkoxy, (C₂-C₈) alkanoyl, lower(C₁-C₄) alkyl, lower(C₁-C₄) alkyl substituted by one or more hydroxy group, lower(C₁-C₄) alkoxy, nitro, cyano, hydroxy, 1,2,4-triazolyl, 1,3-imidazolyl, 1,2,3,4-tetrazolyl.

The more preferred compounds of the present invention are the compounds of Formula II 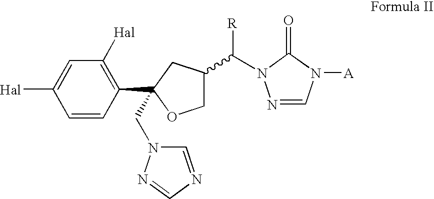
(Formula I, wherein: Az is 1,2,4-triazo-1-yl; R is H, CH₃; Ar is 2,4-dihalo substituted phenyl,
and R₁ is 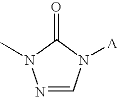
wherein A is the same as defined earlier and preferred A is 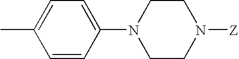
wherein
Z is a hydrogen, (C₁-C₈) alkanoyl, lower alkyl, (C₁-C₈) perhaloalkanoyl, or phenyl, phenyl substituted by one or more of groups independently selected from nitro, cyano, halogen (chlorine, fluorine bromine, iodine) perhalo lower(C₁-C₄) alkyl, perhalo lower(C₁-C₄) alkoxy,(C₂-C₈) alkanoyl, lower(C₁-C₄) alkyl, lower (C₁-C₄) alkyl substituted by one or more hydroxy group, lower(C₁-C₄) alkoxy, 1,3-imidazolyl, 1,2,4-triazolyl, 1,2,3,4-tetrazolyl, or OCH₂Y; wherein
Y is phenyl or phenyl substituted by one or more of groups independently selected from nitro, cyano, halo, perhalo lower alkyl, (C₂-C₈) alkanoyl lower alkyl, hydroxy, lower alkyl substituted by one or more hydroxy group, lower alkoxy, 1,3-imidazolyl, 1,2,4-triazolyl or 1,2,3,4-tetrazolyl; hal is selected from the group consisting of chlorine, fluorine, bromine and iodine atoms and preferred halo is fluorine atom.

Pharmaceutically acceptable salts are non toxic acid addition salts, formed by adding inorganic or organic acids to the compounds of the present invention, by methods well known in the art.

It is also an object of the invention to provide a method for synthesis of the novel compounds.

The present invention also relates to a method of treating or preventing fungal infections in a mammal by administering to said mammal compositions containing the compounds of the present invention.

The present invention also includes within its scope prodrugs of Formulae I and II. In general, such prodrugs will be functional derivatives of the compound which readily get converted in vivo into defined compounds. Conventional procedures for the selection and preparation of suitable prodrugs are known.

Other advantages of the invention will be set forth in the description which follows, and in part will be apparent from the description or may be learned by the practice of the invention.

The illustrated list of compounds of Formula I include
2-[(5R,3R)-5-(2,4-Difluorophenyl)-tetrahydro-5-(1H-1,2,4-triazol-1yl-methyl) furan-3-yl-methyl]-4-{4-[4-(phenyl)-1-piperazinyl]-chlorophenyl}-2,4-dihydro-3(2H,4h)-1,2,4-triazolone (Compound No. 1),
2-[(5R,3S)-5-(2,4-Difluorophenyl)-tetrahydro-5-(1H-1,2,4-triazol-1yl-methyl)-furan-3-yl-methyl]-4-[4-(phenyl)-1,2,4-triazol-1-yl]-2,4-dihydro-3(2H,4H)-1,2,4-triazolone (Compound No. 2),
2-[(5R,3S)-5-(2,4-Diflurophenyl)-tetrahydro-5-(1H-1,2,4-triazol-1yl-methyl)-furan-3-yl-methyl]-4-[4-(hydroxyphenyl)-2,4-dihydro-3(2H,4H)-1,2,4-triazolone (Compound No. 3),
2-[(5R,3R)-5-(2,4-Difluorophenyl)-tetrahydro-5-(1H-1,2,4-triazol-1yl-methyl)-furan-3-yl-methyl]4-[4-(1,2,4-triazol-1-yl-methyl)-phenyl-2,4-dihydro-3(2H,4H)-1,2,4-triazolone (Compound No. 4),
2-[(5R,3S)-5-(2,4-Difluorophenyl)-tetrahydro-5-(1H-1,2,4-triazol-1yl-methyl)-furan-3-yl-methyl]-4-{4-[4-(phenyl)-1-piperazinyl]-chlorophenyl}-2,4-dihydro-3(2H,4H-1,2,4-triazolone (Compound No. 5),
2-[(5R,3S)-5-(2,4-Difluorophenyl)-tetrahydro-5-(1H-1,2,4-triazol-1yl-methyl)-furan-3-yl-methyl]-4-[4-(benzyloxy)-phenyl]-2,4-dihydro-3(2H,4H)-1,2,4-triazolone (Compound No. 6),
2-[(5R,3S)-5-(2,4-Difluorophenyl)-tetrahydro-5-(1H-1,2,4-triazol-1yl-methyl)-furan-3-yl-methyl]-4-{4-[4-[4-(benzyloxy)-phenyl]-1-piperazinyl]-phenyl}-2,4-dihydro-3(2H,4H)-1,2,4-triazolone (Compound No. 7),
2-[(5R,3R)-5-(2,4-Difluorophenyl)-tetrahydro-5-(1H-1,2,4-triazol-1yl-methyl)-furan-3-yl-methyl]-4-[4-(2,2,3,3-tetrafluoropropoxy)-phenyl]-2,4-dihydro-3(2H,4H-1,2,4-triazolone (Compound No. 8),
2-[(5R,3R)-5-(2,4-Difluorophenyl)-tetrahydro-5-(1H-1,2,4-triazol-1yl-methyl)-furan-3-yl-methyl]-4-[4-(1,2,3,4-tetrazol-1-yl)-phenyl]-2,4-dihydro-3(2H,4H)-1,2,4-triazolone (Compound No. 9),
2-[(5R,3S)-5-(2,4-Difluorophenyl)-tetrahydro-5-(1H-1,2,4-triazol-1yl-methyl)-furan-3-yl-methyl]-4-[4-(2,4-dichlorobenzyloxy)-phenyl]-2,4-dihydro-3(2H,4H)-1,2,4-triazolone (Compound No. 10),
2-[(5R,3R)-5-(2,4-Difluorophenyl)-tetrahydro-5-(1H-1,2,4-triazol-1yl-methyl)-furan-3-yl-methyl]-4-{4-[4-[4-(benzyloxy)-phenyl]-1-piperazinyl]-phenyl}-2,4-dihydro-3(2H,4H)-1,2,4-triazolone (Compound No. 11),
2-[(5R,3R)-5-(2,4-Difluorophenyl)-tetrahydro-5-(1H-1,2,4-triazol-1yl-methyl)-furan-3-yl-methyl]-4-[4-(2,4-dichlorobenzyloxy)-phenyl]-2,4-dihydro-3(2H,4H-1,2,4-triazolone (Compound No. 12).

### DETAILED DESCRIPTION OF THE INVENTION

In order to achieve the above mentioned objectives and in accordance with the purpose of the invention as embodied and broadly described herein, there is provided a process for the synthesis of compound of Formula I and Formula II, as shown in Schemes I and II.

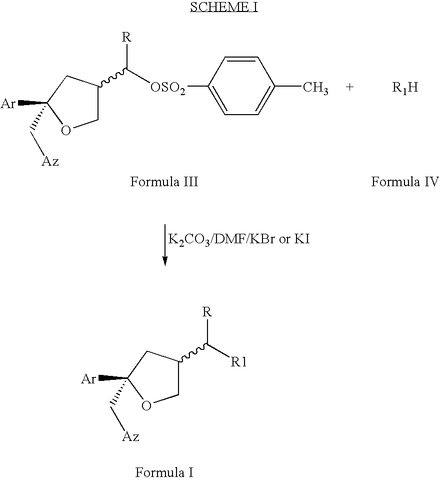

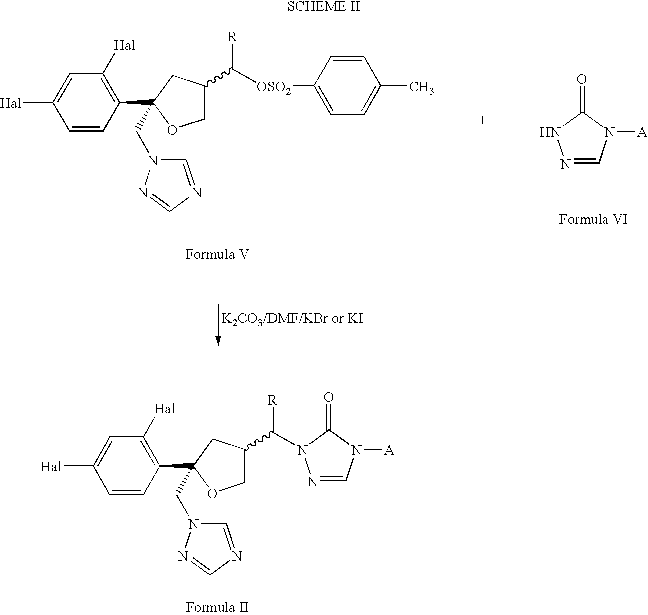

In scheme I, there is provided a process for preparing a compound of Formula I, which comprises reacting a compound of Formula III with a compound of Formula IV wherein Az is a five to seven membered heterocyclic ring having one to four heteroatoms selected from N, S, or O; the preferred heterocyclic ring is 1,2,4-triazol-1-yl.

Ar is a five to seven membered heterocyclic ring containing one to four heteroatoms selected from the group consisting of oxygen nitrogen and sulphur; phenyl or a substituted phenyl group having one to three substituents independently selected from halogen (e.g. chlorine, fluorine, bromine or iodine), nitro, cyano, lower (C₁-C₄) alkyl, lower (C₁-C₄) alkoxy or perhalo lower (C₁-C₄) alkyl, perhalo lower (C₁-C₄) alkoxy; the preferred heterocyclic rings are thienyl and pyridyl;
R is H or methyl;
R₁ is selected from the group consisting of 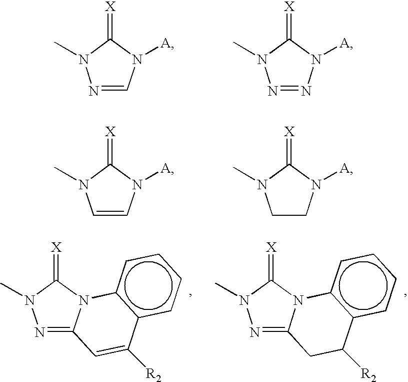
wherein
X is selected from the group consisting of CH₂, O, S and SO₂;
R₂ is hydrogen or lower (C₁-C₄) alkyl;
A is hydrogen, lower (C₁-C₄) alkyl, phenyl or phenyl substituted by one or more of groups independently selected from halogen (e.g. chlorine, fluorine, bromine or iodine atom), nitro, cyano, hydroxy, lower(C₁-C₄) alkyl, lower(C₁-C₄) alkoxy, perhalo lower(C₁-C₄)alkoxy or perhalo lower (C₁-C₄) alkyl, substituted or unsubstituted five or six membered heterocyclyl ring system containing one to four hetero atoms chosen from N, O and S, said heterocyclyl substituents being (C₁-C₈) alkanoyl, lower (C₁-C₄) alkyl, lower (C₁-C₄) alkoxy carbonyl, N,N-di(lower alkyl) (C₁-C₄) amino carbonyl, aminothiocarbonyl, N-lower(C₁-C₄) alkyl aminothiocarbonyl, N,N-di(lower alkyl) (C₁-C₄) aminothiocarbonyl, lower (C₁-C₄) alkyl sulfonyl, phenyl substituted lower (C₁-C₄) alkyl sulfonyl, N-lower(C₁-C₄) alkylamino, N,N-di(lower alkyl) (C₁-C₄) amino, 1,3-imidazol-1-yl, 2-loweralkyl(C₁-C₄) sulfenyl-1,3-imidazol-1-yl, pyridinyl, thiazolyl, 1,2,4 triazol-4-yl or phenyl or phenyl substituted by one or more of groups independently selected from halogen (chlorine, fluorine, bromine or iodine), perhalo lower(C₁-C₄) alkyl, perhalo lower(C₁-C₄) alkoxy, (C₂-C₈) alkanoyl, lower(C₁-C₄) alkyl, lower(C₁-C₄) alkyl substituted by one or more hydroxy group, lower(C₁-C₄) alkoxy, nitro, cyano, hydroxy, 1,2,4-triazolyl, 1,3-imidazolyl, 1,2,3,4-tetrazolyl.

The starting compound of general Formula III can be prepared by the processes as described in the U.S. Pat. Nos. 5,661,151; 5,703, 236; and 5,039,676. The starting compound of general Formula IV can be prepared by the processes as described in the U.S. Pat. Nos. 5,371,101 and 6,034,248; Chem. Ber. 1970; 103:1960 and Chem. Ber. 1975; 108:3799. These starting compounds for Scheme I may be suitably adapted using these references to produce the compounds of Formula I.

The reaction of compound of Formula III with the compound of Formula IV may be carried out in the presence of a suitable base selected from the group consisting of sodium hydride, sodium carbonate, potassium carbonate, cesium carbonate and the like. The reaction may be carried out in the presence of solvents like dimethylformamide, dimethyl sulfoxide, toluene, isopropyl alcohol, tetrahydrofuran, ethylene glycol, dimethyl ether (DME), and the like, or mixtures thereof. The reaction temperature may range from 30°-120° C., preferably at a temperature in the range of 80°-85° C.

Scheme II shows the synthesis of compounds of the Formula II in which R, A and Halo groups are as defined above.

The preparation comprises condensing 2,2,4-trisubstituted tetrahydrofuran of the Formula V with 4-substitued triazolone of the Formula VI, wherein A is the same as defined before, in the presence of a base and an organic solvent like dimethylformamide, at a temperature ranging from 30-125° C. and preferably at 80-85° C., for a period varying between one to several hours to produce the corresponding 1,4-disubstituted triazolones of the Formula II.

In the above schemes, where specific bases and solvents, etc. are mentioned, it is understood that other bases, and solvents known to those skilled in the art may also be used. Similarly, the reaction temperature and duration of the reactions may be adjusted according to the desired needs.

### PHARMACOLOGICAL ACTIVITY

Compound of the Formula I and its salts are useful in the curative or prophylactic treatment of fungal infections in animals, including human.

The in vitro evaluation of the antifungal activity of the compound of this invention (as shown in Table I) can be performed by determining the minimum inhibitory concentration (MIC) which is the concentration of the test compound in Rosewell Park Memorial Institute (RPMI) 1640 liquid medium buffered with 3-(Morpholino) propane sulfonic acid (MOPS) to pH 7, at which there is significant inhibition of the particular fungi. In practice the National Committee for Clinical Laboratory Standard (NCCLS) M27A document for Candida and Cryptococcus and M38P for Aspergillus was used to determine the MIC were determined and readings recorded only when the Quality Control results fell into the acceptable range. After MIC results had been recorded, 100 μL from each of the well showing no growth was spread over Sabouraud Dextrose Agar (SDA) to determine the minimum fungicidal concentration (MFC).

The in vivo evaluation of the compound can be carried out at a series of dose levels by oral or i.v. injection to mice which are inoculated I.V. with the minimum lethal dose of Candida albicans, Cryptococcus neoformans or Aspergillus fumigatus by the tail vein. Activity is based on the survival of a treated group of mice after the death of an untreated group of mice. For Aspergillus and Cryptococcus infections, target organs were cultured after treatment to document the number of mice cured of the infection for further assessment of activity.

For human use, the antifungal compound of the present invention and its salts can be administered above, but will generally by administered in admixture with a pharmaceutical carrier selected with regard to the intended route of administration and standard pharmaceutical practice for example, they can be administered orally in the form of tablets containing such exciepients as starch or lactose or in capsules or ovules either alone or in admixture with exciepients or in the form of elixirs, solutions or suspensions containing flavoring or coloring agents. They can be injected parenterally, for example, intravenously, intramuscularly or sub-cutaneously. For parenteral administration they are best used in the form of a sterile aqueous solution which may contain other substances, for example, enough salts or glucose to make the solution isotonic with blood.

TABLE I

| | MIC of Compounds (g/ml) | | | | | | | |
|---|---|---|---|---|---|---|---|---|
| Organization | Compound No. | 4 | 2 | 6 | 3 | 5 | 7 | 9 |
| Candida parapsilosis ATCC | <0.00025 | 8 | 8 | 0.25 | 16 | 0.03 | 0.125 | 16 |
| 22019 (QC) | | | | | | | | |
| Candida brusei ATCC 6258 | 0.125 | 16 | 8 | 4 | 16 | 0.25 | 8 | 64 |
| (QC) | | | | | | | | |
| Paecilomyces variotti ATCC | Ng | 16 | 8 | 16 | >16 | 1 | 1 | 32 |
| 22319 (QC) | | | | | | | | |
| Cryptococcus neoformans I | 0.004 | 8 | 8 | 2 | 8 | <0.03 | 0.06 | 32 |
| Cryptococcus neoformans M | 0.016 | 8 | 8 | 2 | 8 | 0.125 | 0.125 | 8 |
| 106 | | | | | | | | |
| Histoplasma capsulatum | 0.03 | 16 | 16 | 0.5 | 16 | 0.25 | 16 | 64 |
| Candida tropicalis ATCC 750 | 0.002 | 16 | 0.5 | 0.06 | 16 | 0.03 | 0.125 | 8 |
| Candida krusel 766.1 | 0.125 | 16 | 16 | 8 | 16 | 1 | 16 | 64 |
| Candida albicans Y-01-19 | Ng | 16 | 16 | 1 | 8 | 0.125 | 16 | 64 |
| Candida albicans ATCC 36082 | 0.03 | 2 | 0.125 | 0.03 | 16 | 0.03 | <0.03 | |
| Candida glabrata 90030 | 0.5 | 16 | 16 | 2 | 16 | 1 | 16 | 64 |
| Aspergillus fumigatus 1008 | 0.25 | 16 | 16 | 16 | >16 | 2 | 16 | >128 |
| Aspergillus fumigatus Si-I | 0.25 | 16 | 16 | 16 | >16 | 1 | 16 | ≧128 |
| Candida albicans 1122 | — | — | — | — | — | — | — | 4 |
| Candida albicans 1162 | — | — | — | — | — | — | — | 64 |

The invention is explained in detail in the examples given below which are provided by way of illustration only and therefore should not be constrained to limit the scope of the invention.

### EXAMPLE 1

### Preparation of 2-[(5R,3R)-5-(2,4-difluorophenyl)-tetrahydro-5-(1H-1,2,4-triazol-1yl-methyl)-furan-3-yl-methyl]-4-{4-[4-(phenyl)-1-piperazinyl]-chlorophenyl}-2,4-dihydro-3(2H,4H-1, 2,4-triazolone

A mixture of (3R,5R)-5-(2,4-difluorophenyl)-5-[(1H-1,2,4-triazol-1-yI)-methyl]-tetrahydro-3-furanmethanol, 4-toluenesulfonate (0.25 gm, 0.556 mmol) and potassium bromide (0.132 gm, 1.113 mmol) in DMF (15 ml) was heated at 80-85° C. for 1 hour. Potassium carbonate (0.154 g, 1.113 mmol) and 4-{4-[4-(chlorophenyl)-1-piperazinyl]-phenyl}-3(2H,4H)-1,2,4-triazolone (0.178 gm, 0.50 mmol) were added to the above mixture at room temperature and the resultant mixture was further heated at 80-85° C. for 5 hours. After the reaction was over, the mixture was poured over crushed ice and extracted with ethyl acetate (3×50 ml). The combined organic layers were washed with water (3×100 ml), and brine (50 ml) successively, then dried over anhydrous sodium sulphate, filtered and evaporated in vacuo to afford an oily residue. Chromatographed the residue on silica gel, eluting with hexane-ethyl acetate (9:1), to afford the title compound as white solid. Yield: 0.285 g, (81%).

¹HNMR (CDCl₃): δ8.11(1H, s, triazole-H), 7.77(1H, s, triazole-H), 7.58(1H, s, triazolone-H), 7.41-7.33(2H, m, Ar—H), 7.41-7.33(2H, m, Ar—H), 7.33(1H, m, Ar—H), 7.25-7.22(2H, m, Ar—H), 7.02(2H, d, J=8.94 Hz, Ar—H), 6.82-6.78(2H, m, Ar—H), 4.66-4.53(2H, dd, J=14.37 & 14.49 Hz, CH₂-triazole), 4.13-4.07(1H, m, CH₂-triazolone), 3.90-3.83(1H, m, CH₂-triazolone), 3.79-3.68(2H, m, C-2H), 3.36-3.30(8H, m, piperazine-H), 2.64-2.53(2H, m, C-4H & C-3H) and 2.08-2.00(1H, m, C-4H)

IR(KBr): 3445, 2835, 1699(CO), 1498 and 1230 cm⁻¹

MS(positive ion mode) m/z: 633.3 [M⁺+1]

m.p.: 171-175° C.

### EXAMPLE 2

### Preparation of 2-[(5R,3S)-5-(2,4-difluorophenyl)-tetrahydro-5-(1H-1,2,4-triazol-1yl-methyl)-furan-3-yl-methyl]-4-[4-(phenyl)-1,2,4triazol-1-yl]-2,4-dihydro-3(2H,4H)-1,2,4-triazolone

By following the procedure of Example 1 and reacting (3S,5R)-5-(2,4-difluorophenyl)-5-[(1H-1,2,4-triazol-1-yl)-methyl]-tetrahydro-3-furanmethanol, 4-toluene sulfonate and 4-[4-(phenyl)-1,2,4-triazol-1-yl]-3(2H,4H)-1,2,4-triazolone afforded the title compound.

¹HNMR (CDCl₃): δ8.59(1H, s, triazole-H), 8.13(1H, s, triazole-H), 8.08(1H, s, triazolone-H), 7.85-7.78(3H, m, Ar—H), 7.69-7.68(3H, m, Ar—H), 7.51-7.43(1H, m, Ar—H), 6.89-6.80(2H,m, Ar—H), 4.56(1H, d, J=14.25 Hz, CH₂-triazole), 4.35(1H, d, J=14.25 Hz, CH₂-triazole), 4.14-4.08(1H, m, CH₂-triazolone), 3.81-3.60(3H, m, CH₂-triazolone & C-2H), 2.83-2.75(1H, m, C-3H), 2.34-2.24(1H, m, C-4) and 2.13-2.06(1H, m, C-4H)

IR(KBr): 3442, 1695(CO), 1529, 1402 and 1276 cm⁻¹

MS(positive ion mode) m/z: 506.1 [M⁺+1]

m.p.: 186-187° C.

### EXAMPLE 3

### Preparation of 2-[(5R,3S)-5-(2,4-diflurophenyl)-tetrahydro-5-(1-1S1,2,4-triazol-1yl-methyl)-furan-3-yl-methyl]-4-[4-(hydroxyphenyl)]-2,4-dihydro-3(2H,4)-1,2,4-triazolone

The title compound was prepared by an analogous procedure to that described in Example 1 using (3S,5R)-5-(2,4-difluorophenyl)-5-[(1H-1,2,4-triazol-1-yl)-methyl]-tetrahydro-3-furanmethanol, 4-toluene sulfonate and 4-[4-(hydroxyphenyl)]-3(2H,4H)-1,2,4-triazolone.

¹HNMR (CDCl₃+MeOD): δ 8.19(1H, s, triazole-H), 7.81(1H, s, triazole-H), 7.62(1H, s, triazolone-H), 7.45-7.40(2H, m, Ar—H), 6.92-6.79(4H, m, Ar—H), 4.56(1H, d, J=14.23 Hz, CH₂triazole), 4.45(1H, d, J=14.23 Hz, CH₂-triazole), 4.17-4.12(1H, m, CH₂-triazolone), 3.80-3.61(3H, m, C-2H & CH₂-triazolone), 3.36(1H, brs, —OH), 2.78-2.71(1H, m, C-3h), 2.50-2.42(1H, m, C-4H) and 2.16-2.10(1H, m, C-4H)

IR(KBr): 3449(OH), 1684(CO), 1515 and 1274 cm⁻¹

MS(positive ion mode) m/z: 454 [M⁺+1]

m.p.: 199.1-201.4° C.

### EXAMPLE 4

### Preparation of 2-[(5R,3R)-5-(2,4-difluorophenyl)-tetrahydro-5-(1H-1,2,4-triazol-1yl-methyl)-furan-3-yl-methyl]-4-[4-(1,2,4-triazol-1-yl-methyl)-phenyl-2,4-dihydro-3(2H,4)-1,2,4-triazolone

The title compound was prepared by an analogous procedure to that described in Example 1 using (3R,5R)-5-(2,4-difluorophenyl)-5-[(1H-1,2,4-triazol-1-yl)-methyl]-tetrahydro-3-furanmethanol, 4-toluene sulfonate and 4-[4-(1,2,4-triazol-1-yl-methyl)-phenyl]-3(2H,4H)-1,2,4-triazolone afforded the title compound.

¹HNMR (CDCl₃): δ 8.15(1H, s, triazole-H), 8.00(1H, s, triazole-H), 7.96(1H, s, triazolone-H), 7.80(1H, s, triazole-H), 7.78(1H, s, triazole-H), 7.68-7.56(3H, m, Ar—H), 7.44-7.37(2H, m, Ar—H, 6.84-6.78(1H, m, Ar—H), 5.39(2H, m, CH₂-triazolone & CH₂-triazole), 5.09-4.98(2H, m, CH₂-triazole), 4.61-4.57(1H, m, C-2H), 4.13-4.07(1H, m, C-2H), 4.10(1H, d, J=5.00 Hz, CH₂-triazole), 3.84-3.72(1H, m, C-2H) and 2.09-2.04(3H, m, C-2H & C-4H)

IR(KBr): 3431, 1706(CO), 1503 and 1273 cm⁻¹

MS(positive ion mode) m/z: 520 [M⁺+1]

m.p.: 60-62.7° C.

### EXAMPLE 5

### Preparation of 2-[(5R,3S)-5-(2,4-difluorophenyl)-tetrahydro-5-(1H-1,2,4-triazol-1yl-methyl)-furan-3-yl-methyl]4-{4-[4-(phenyl)-1-piperazinyl]-chlorophenyl}-2,4-dihydro-3(2H,4H)-1,2,4-triazolone

The title compound was prepared by an analogous procedure to that described in Example 1 using (3S,5R)-5-(2,4-difluorophenyl)-5-[(1H-1,2,4-triazol-1-yl)-methyl]-tetrahydro-3-furanmethanol, 4-toluene sulfonate and 4-{4-[4-(phenyl)-1-piperazinyl]-chlorophenyl}-3(2H,4H-1,2,4-triazolone afforded the title compound.

¹HNMR (CDCl₃): δ 8.10(1H, s, triazole-H), 7.85(1H, s, triazole-H), 7.54(1H, s, triazolone-H), 7.49-7.41 (1H, m, Ar—H), 7.36(2H, d, J=10.75 Hz, Ar—H), 7.26-7.23(2H, m, Ar—H), 7.01(2H, d, J=8.8 Hz, Ar—H), 6.91-6.80(4H, m, Ar—H), 4.56(1H, d, J=14.23 Hz, CH₂-triazole), 4.32(1H, d, J=14.23 Hz, CH₂-triazole), 4.14-4.09(1H, m, CH₂-triazolone ), 3.78-3.70(2H, m, C-2H & CH₂-triazolone), 3.65-3.58(1H, m, C-2H), 3.35-3.32(8H, brm, piperazine-H), 2.81-2.74(1 H, m, C-3H), 2.37-2.28(1H, m, C-3H & C-4H) and 2.13-2.06(1H, m, C-4H)

IR(KBr): 2833, 1691 (CO), 1520, 1498 and 1232 cm⁻¹

MS(positive ion mode) m/z: 633.2 [M⁺+1]

m.p.: 177-178.2° C.

### EXAMPLE 6

### Preparation of 2-[(5R,3S)-5-(2,4-difluorophenyl)-tetrahydro-5-(1H-1,2,4-triazol-1yl-methyl)-furan-3-yl-methyl]-4-[4-(benzyloxy)-phenyl]-2,4-dihydro-3(2H,4M-1,2,4-triazolone

The title compound was prepared by an analogous procedure to that described in Example 1 using (3S,5R)-5-(2,4-difluorophenyl)-5-[(1H-1,2,4-triazol-1-yl)-methyl]-tetrahydro-3-furanmethanol, 4-toluene sulfonate and 4-[4-(benzyloxy)-phenyl]-3(2H,4H)-1,2,4-triazolone afforded the title compound.

¹HNMR (CDCl₃): δ 8.09(1H, s, triazole-H), 7.58(1H, s, triazole-H), 7.53(1H, s, triazolone-H), 7.46-7.35(8H, m, Ar—H), 7.06-7.03(2H, m, Ar—H), 6.88-6.80(2H, m, Ar—H), 5.09(2H, s, OCH₂), 4.55(1H, d, J=14.27 Hz, CH₂-triazole), 4.36(1H, d, J=14.23 Hz, CH₂-triazole), 4.13-4.08(1H, m, CH₂-triazolone), 3.79-3.69(2H, m, C-2H & CH₂-triazolone), 3.65-3.60(1H, m, C-2H), 2.77-2.75(1H, m, C-3H), 2.13-2.09(1H, m, C-4H) and 2.07-2.05(1H, m, C-4H)

IR(KBr): 3434, 1691 (CO), 1517 and 1255 cm⁻¹

Ms(positive ion mode) m/z: 545 [M⁺+1]

m.p.: 128.2-131.7° C.

### EXAMPLE 7

### Preparation of 2-[(5R,3S)-5-(2,4-difluorophenyl)-tetrahydro-5-1H-1,2,4-triazol-1yl-methyl)-furan-3-yl-methyl]-4-{4-[4-[4-benzyloxy)-phenyl]-1-piperazinyl]-phenyl}-2,4-dihydro-3(2H,4H)-1,2,4-triazolone

The title compound was prepared by an analogous procedure to that described in Example 1 using (3S,5R)-5-(2,4-difluorophenyl)-5-[(1H,1,2,4-triazol-1-yl)-methyl]-tetrahydro-3-furanmethanol, 4-toluene sulfonate and 4-[4-[4-[4-(benzyloxy)-phenyl]-1-piperazinyl]-phenyl]-3(2H,4H)-1,2,4-triazolone afforded the title compound.

¹HNMR (CDCl₃): δ 8.08(1H, s, triazole-H), 7.84(1H, s, triazole-H), 7.46(1H, s, triazolone-H), 7.44-7.32(9H, m, Ar—H), 7.03-6.94(4H, m, Ar—H), 6.85-6.82(3H, m, Ar—H), 5.03(2H, s, OCH₂), 4.57(1H, d, J=14.23 Hz, CH₂-triazole), 4.38(1H, d, J=14.26 Hz, CH₂-triazole), 4.18-4.08(1H, m, CH₂-triazolone ), 3.75-3.62(3H, m, C-2H & CH₂-triazolone), 3.38-3.35(4H, m, piperazine-H), 3.25-3.23(4H, m, piperazine-H), 2.76-2.60(1H, m, C-3H), 2.53-2.31 (1H, m, C-4m and 2.12-1.96(1H, m, C-4H)

IR(KBr): 3448, 2930, 1693(CO), 1516 and 1271 cm⁻¹

MS(positive ion mode) m/z: 705 [M⁺+1]

m.p.: 166.4-167.8° C.

### EXAMPLE 8

### Preparation of 2-[(5R,3R)-5-(2,4-difluorophenyl)-tetrahydro-5-(1H-1,2,4-triazol-1yl-methyl)-furan-3-yl-methyl]-4-[4-(2,2,3,3-tetrafluoropropoxy)-phenyl]-2,4-dihydro-3(2H,4H)-1,2,4-triazolone

The title compound was prepared by an analogous procedure to that described in Example 1 using (3R,5R)-5-(2,4-difluorophenyl)-5-[(1H-1,2,4-triazol-1-yl)-methyl]-tetrahydro-3-furanmethanol, 4-toluene sulfonate and 4-[4-(2,2,3,3-tetrafluoropropoxy)-phenyl]-3(2H,4H)-1,2,4-triazolone afforded the title compound.

¹HNMR (CDCl₃): δ 8.29(1H, s, triazole-H), 7.83(1H, s, triazole-H), 7.62(1H, s, triazolone-H), 7.49(2H, d, J=8.70 Hz, Ar—H), 7.32-7.29(1H, m, Ar—H), 7.03(2H, d, J=8.70 Hz, Ar—H), 6.86-6.80(2H, m, Ar—H), 6.23-5.88(1H, ttt, CHF₂), 4.65-4.57(1H, m, CH₂-triazole), 4.42-4.34(1H, m, CH₂-triazole), 4.13-4.08(1H, m, CH₂-triazolone), 3.91-3.74(3H, m, C-2H & CH₂-triazolone), 2.65-2.51(2H, m, C-3H & C-4H) and 2.08-2.01 (1H, m, C-4H)

IR(KBr): 3446,1706(CO), 1517,1136 and 1108 cm⁻¹

MS(positive ion mode) m/z: 568 [M⁺+1]

m.p.: 64.5-66.4° C.

### EXAMPLE 9

### Preparation of 2-[(5R,3R)-5-(2,4-difluorophenyl)-tetrahydro-5-(1H-1,2,4-triazol-1yl-methyl)-furan-3-yl-methyl]-4-[4-(1,2,3,4-tetrazol-1-yl)-phenyl]-2,4-dihydro-3(2H,4H)-1,2,4-triazolone

The title compound was prepared by an analogous procedure to that described in Example 1 using (3R,5R)-5-(2,4-difluorophenyl)-5-[(1H-1,2,4-triazol-1-yl)-methyl]-tetrahydro-3-furanmethanol, 4-toluene sulfonate and 4-[4-(1,2,3,4-tetrazol-1-yl)-phenyl]-3(2H,4H)-1,2,4-triazolone afforded the title compound.

¹HNMR (CDCl₃): δ 8.14(1H, s, triazole-H), 7.80(1H, s, triazole-H), 7.61(1H, s, triazolone-H), 7.44-7.34(3H, m, Ar—H), 7.05(2H, d, J=8.42 Hz, Ar—H), 6.87-6.78(2H, m, Ar—H), 4.66 (1H, d, J=14.48 Hz, CH₂-triazole), 4.54(1H, d, J=14.35 Hz, CH₂-triazole), 4.12-4.07(1H, m, CH₂-triazolone), 3.82-3.69(3H, m, C-2H & CH₂-triazolone), 2.64-2.54(2H, m, C-3H & C4H) and 2.08-2.01 (1 H, m, C-4H)

IR(KBr): 3490, 2927, 1707(CO), 1521,1407,1270 and 1137 cm⁻¹

MS(positive ion mode) m/z: 479 [M⁺+1]

m.p.: 73.7-75.2° C.

### EXAMPLE 10

### Preparation of 2-[(5R,3S)-5-(2,4-difluorophenyl)-tetrahydro-5-(1H-1,2,4-triazol-1yl-methyl)-furan-3-yl-methyl][4-(2,4-dichlorobenzyloxy)-phenyl]-2,4-dihydro-3(2H,4H)-1,2,4triazolone

The title compound was prepared by an analogous procedure to that described in Example 1 using (3S,5R)-5-(2,4-difluorophenyl)-5-[(1H-1,2,4-triazol-1-yl)-methyl]-tetrahydro-3-furanmethanol, 4-toluene sulfonate and 4-[4-(2,4-dichlorobenzyloxy)-phenyl]-3(2H,4H)-1,2,4-triazolone afforded the title compound.

¹HNMR (CDCl₃): δ 8.09(1H, s, triazole-H), 7.84(1H, s, triazole-H), 7.56(1H, s, triazolone-H), 7.54-7.38(5H, m, Ar—H), 7.30(1H, m, Ar—H), 7.05-7.03(2H, m, Ar—H), 6.88-6.83(2H, m, Ar—H), 5.14(2H, s, OCH₂), 4.55(1H, d, J=14.38 Hz, CH₂-triazole), 4.36(1H, d, J=14.23 Hz, CH₂-triazole), 4.13-4.08(1H, m, CH₂-triazolone), 3.79-3.57(3H, m, C-2H & CH₂-triazolone), 2.80-2.73(1H, m, C-3H), 2.33-2.29(1H, m, C-4H) and 2.12-2.05(1H, m, C-4H)

IR(KBr): 3448, 2929, 1707(CO), 1515, 1246 and 1137 cm⁻¹

MS(positive ion mode) m/z: 613 [M⁺+1]

m.p.: 100.7-104.7° C.

### EXAMPLE 11

### Preparation of 2-[(5R,3R)-5-(2,4-difluorophenyl)-tetrahydro-5-(1H-1,2,4-triazol-1yl-methyl)-furan-3-yl-methyl]-4-{4-[4-[4-(benzyloxy)-phenyl]-1-piperazinyl]-phenyl}-2,4-dihydro-3(2H,4H)-1,2,4-triazolone

The title compound was prepared by an analogous procedure to that described in Example 1 using (3R,5R)-5-(2,4-difluorophenyl)-5-[(1H-1,2,4-triazol-1-yl)-methyl]-tetrahydro-3-furanmethanol, 4-toluene sulfonate and 4-{4-[4-[4-(benzyloxy)-phenyl]-1-piperazinyl]-phenyl}-3-(2H,3H)-1,2,4-triazolone afforded the title compound.

¹HNMR (CDCl₃): δ 8.11(1H, s, triazole-H), 8.09(1H, s, triazole-H), 7.77(1H, s, triazolone-H), 7.45-7.32(8H, m, Ar—Hh), 7.04-6.95(6H, m, Ar—H), 6.90-6.79(2H, m, Ar—H), 5.04(2H, s, OCH₂), 4.62-4.59(2H, m, CH₂-triazole), 4.11 (1H, m, CH₂-triazolone), 3.86-3.73(3H, m, C-2H & CH₂-triazolone), 3.38-3.35(4H, brm, piperazine-H), 3.25-3.23(4H, brm, piperazine-H), 2.35-2.25(2H, m, C-3H & C-4H) and 2.09-2.04(1H, m, C-4H)

IR(KBr): 3421, 2827, 1695(CO), 1516 and 1249 cm⁻¹

MS(positive ion mode) m/z: 705 [M⁺+1]

m.p.: 174.5-178.5° C.

### EXAMPLE 12

### Preparation of 2-[(5R,3R)-5-(2,4-difluorophenyl)-tetrahydro-5-(1H-1,2,4-triazol-1yl-methyl)-furan-3-yl-methyl]-4-[4-(2,4-dichlorobenzyloxy)-phenyl]-2,4-dlhydro-3(2H,4H)-1,2,4-triazolone

The title compound was prepared by an analogous procedure to that described in Example 1 using (3R,5R)-5-(2,4-difluorophenyl)-5-[(1H-1,2,4-triazol-1-yl)-methyl]-tetrahydro-3-furanmethanol, 4-toluene sulfonate and 4-[4-(2,4-dichlorobenzyloxy)-phenyl]-3(2H,4H-1,2,4-triazolone afforded the title compound.

¹HNMR (CDCl₃): δ 8.14(1H, s, triazole-H), 7.81(1H, s, triazole-H), 7.62(1H, s, triazolone-H), 7.52-7.45(4H, m, Ar—H), 7.37-7.30(2H, m, Ar—H), 7.08(2H, d, J=8.90 Hz, Ar—H), 6.86-6.82(2H, m, Ar—H), 5.18(2H, s, OCH₂), 4.65-4.56(2H, dd, J=14.43 Hz each, CH₂-triazole), 4.16-4.11(1H, m, CH₂-triazolone), 3.88-3.82(2H, m, C-2H), 3.78-2.76(1H, m, CH₂-triazolone), 2.92-2.57(2H, m, C-3H & C-4H) and 2.11-2.04(1H, m, C-4H)

IR(KBr): 3448, 2930, 1706(CO), 1514, 1246 and 1137 cm⁻¹

MS(positive ion mode) m/z: 613 [M⁺+1]

m.p.: 70-71.2° C.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims without further elaboration, it is believed that one skilled in the art can, using the preceding description utilize the present invention to its fullest extent. Therefore, the examples herein are to be construed as merely illustrative and not limitation of the scope of the present invention in any way. The embodiments of the invention in which an exclusive property or privilege is claimed are defined as follows.

1. A compound having the structure of Formula I, 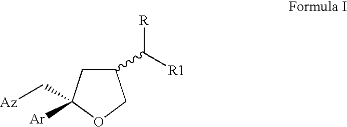
and its pharmaceutically acceptable salts, enantiomers, diastereomers, N-oxides, prodrugs, metabolites, polymorphs, pharmaceutically acceptable solvates,
wherein
Az is a five to seven membered heterocyclic ring having one to four 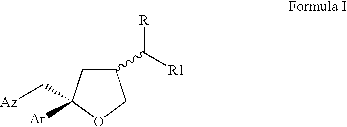
heteroatoms selected from N, S, or O; the preferred heterocyclic ring is 1,2,4-triazol-1-yl;
Ar is a five to seven membered heterocyclic ring containing one to four heteroatoms selected from the group consisting of oxygen nitrogen and sulphur; phenyl or a substituted phenyl group having one to three substituents independently selected from halogen (e.g. chlorine, fluorine, bromine or iodine), nitro, cyano, lower(C₁-C₄) alkyl, lower (C₁-C₄) alkoxy or a perhalo lower (C₁-C₄) alkyl, perhalo lower(C₁-C₄) alkoxy; the preferred heterocyclic rings are thienyl and pyridyl;
R is H or methyl;
R₁ is selected from the group consisting of 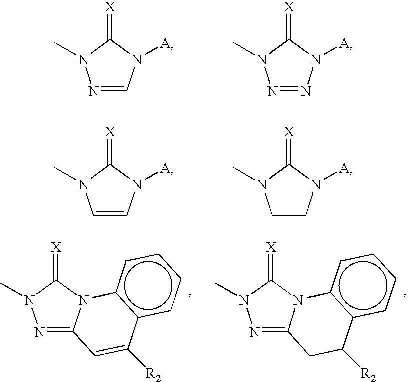
wherein
X is selected from the group consisting of CH₂, O, S and SO₂;
R₂ is hydrogen or lower(C₁-C₄) alkyl;
A is hydrogen, lower(C₁-C₄) alkyl, phenyl or phenyl substituted by one or more of groups independently selected from halogen (e.g. chlorine, fluorine, bromine or iodine atoms ), nitro, cyano, hydroxy, lower(C₁-C₄) alkyl, lower (C₁-C₄) alkoxy or perhalo lower (C₁-C₄) alkyl, perhalo lower (C₁-C₄)alkoxy; substituted or unsubstituted five or six membered heterocyclyl ring systems containing one to four hetero atoms chosen from N, O and S, said heterocyclyl substituents being (C₁-C₈) alkanoyl, lower (C₁-C₄) alkyl, lower (C₁-C₄) alkoxy carbonyl, N,N-di(lower alkyl) (C₁-C₄) amino carbonyl, aminothiocarbonyl, N-lower(C₁-C₄) alkyl aminothiocarbonyl, N,N-di(lower alkyl) (C₁-C₄) aminothiocarbonyl, lower (C₁-C₄) alkyl sulfonyl, phenyl substituted lower (C₁-C₄) alkyl sulfonyl, N-lower (C₁-C₄) alkylamino, N,N-di(lower alkyl) (C₁-C₄) amino, 1,3-imidazol-1-yl, 2-loweralkyl(C₁-C₄) sulfenyl-1,3-imidazol-1-yl, pyridinyl, thaizolyl, 1,2,4 triazol-4-yl or phenyl or phenyl substituted by one or more of groups independently selected from halogen (chlorine, fluorine, bromine or iodine), perhalo lower(C₁-C₄) alkyl, (C₂-C₈) alkanoyl, lower(C₁-C₄) alkyl, lower(C₁-C₄) alkyl substituted by one or more hydroxy group, lower(C₁-C₄) alkoxy, nitro, cyano, hydroxy, 1,2,4-triazolyi, 1,3-imidazolyl, 1,2,3,4-tetrazolyl.

2. The compound of claim 1 having the structure of the Formula II 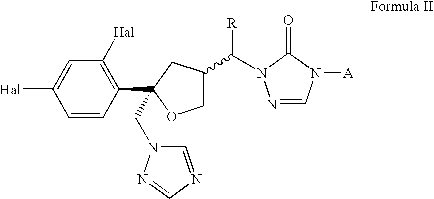
(Formula I, wherein Az is 1,2,4-triazol-1-lyl; R is H or CH₃; Ar is 2,4-dihalo substituted phenyl, Hal is Cl, F, Br or I; and R₁ is 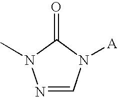
wherein A is the same as defined in claim 1.

3. The compound of claim 2 having the structure of Formula II, wherein A is represented by 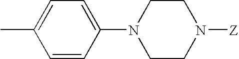
Z is a hydrogen, (C₁-C₈) alkanoyl, lower alkyl, (C₁-C₈) perhaloalkanoyl, or phenyl, phenyl substituted by one or more of groups independently selected from nitro, cyano, halogen (chlorine, fluorine bromine, iodine) perhalo lower(C₁-C₄) alkyl, perhalo lower(C₁-C₄) alkoxy; (C₂-C₈) alkanoyl, lower(C₁-C₄) alkyl, lower (C₁-C₄)alkyl substituted by one or more hydroxy group, lower(C₁-C₄) alkoxy, 1,3-imidazolyl, 1,2,4-triazolyl, 1,2,3,4-tetrazolyl, or OCH₂Y;
wherein Y is phenyl or phenyl substituted by one or more of groups independently selected from nitro, cyano, halo, perhalo lower alkyl, (C₂-C₈) alkanoyl lower alkyl, hydroxy, lower alkyl substituted by one or more hydroxy group, lower alkoxy, 1,3-imidazolyl, 1,2,4-triazolyl or 1,2,3,4-tetrazolyl.

4. A compound selected from the group consisting of:
2-[(5R,3R)-5-(2,4-Difluorophenyl)-tetrahydro-5-(1H-1,2,4-triazol-1yl-methyl)-furan-3-yl-methyl]-4-{4-[4-(phenyl)-1-piperazinyl]-chlorophenyl}-2,4-dihydro-3(2H,4H)-1,2,4-triazolone,
2-[(5R,3S)-5-(2,4-Difluorophenyl)-tetrahydro-5-(1H,1,2,4-triazol-1yl-methyl)-furan-3-yl-methyl]-4-[4-(phenyl)-1,2,4-triazol-1-yl]-2,4-dihydro-3(2H,4H)-1,2,4-triazolone,
2-[(5R,3S)-5-(2,4-Diflurophenyl)-tetrahydro-5-(1H-1,2,4-triazol-1yl-methyl)-furan-3-yl-methyl]-4-[4-(hydroxyphenyl)-2,4-dihydro-3(2H,4H)-1,2,4-triazolone,
2-[(5R,3R)-5-(2,4-Difluorophenyl)-tetrahydro-5-(1H-1,2,4-triazol-1yl-methyl)-furan-3-yl-methyl]-4-[4-(1,2,4-triazol-1-yl-methyl)-phenyl-2,4-dihydro-3(2H,4H)-1,2,4-triazolone,
2-[(5R,3S)-5-(2,4,Difluorophenyl)-tetrahydro-5-(1H-1,2,4-triazol-1yl-methyl)-furan-3-yl-methyl]-4-{4-[4-(phenyl)-1-piperazinyl]-chlorophenyl}-2,4-dihydro-3(2H,4H)-1,2,4-triazolone,
2-[(5R,3S)-5-(2,4-Difluorophenyl)-tetrahydro-5-(1H-1,2,4-triazol-1yl-methyl)-furan-3-yl-methyl]-4-[4-(benzyloxy)-phenyl]-2,4-dihydro-3(2H,4H)-1,2,4-triazolone,
2-[(5R,3S)-5-(2,4-Difluorophenyl)-tetrahydro-5-(1H-1,2,4-triazol-1yl-methyl)-furan-3-yl-methyl]-4-{4-[4-[4-(benzyloxy)-phenyl]-1-piperazinyl]-phenyl}-2,4-dihydro-3(2H,4H)-1,2,4-triazolone,
2-[(5R,3R)-5-(2,4-Difluorophenyl)-tetrahydro-5-(1H-1,2,4-triazol-1yl-methyl)-furan-3-yl-methyl]-4-[4-(2,2,3,3-tetrafluoropropoxy)-phenyl]-2,4-dihydro-3(2H,4H)-1,2,4-triazolone,
2-[(5R,3R)-5-(2,4-Difluorophenyl)-tetrahydro-5-(1H-1,2,4-triazol-1yl-methyl)-furan-3-yl-methyl]4-[4-(1,2,3,4-tetrazol-1-yl)-phenyl]-2,4-dihydro-3(2H,4H)-1,2,4-triazolone,
2-[(5R,3S)-5-(2,4-Difluorophenyl)-tetrahydro-5-(1H-1,2,4-triazol-1yl-methyl)-furan-3-yl-methyl]-4-[4-(2,4-dichlorobenzyloxy)-phenyl]-2,4-dihydro-3(2H,4H)-1,2,4-triazolone,
2-[(5R,3R)-5-(2,4-Difluorophenyl)-tetrahydro-5-(1H-1,2,4-triazol-1yl-methyl)-furan-3-yl-methyl]-4-{4-[4-[4-(benzyloxy)-phenyl]-1-piperazinyl]-phenyl}-2,4-dihydro-3(2H,4H)-1,2,4-triazolone,
2-[(5R,3R)-5-(2,4-difluorophenyl)-tetrahydro-5-(1H-1,2,4-triazol-1yl-methyl)-furan-3-yl-methyl]-4-[4-(2,4-dichlorobenzyloxy)-phenyl]-2,4-dihydro-3(2H,4H)-1,2,4-triazolone,

5. A pharmaceutical composition comprising a therapeutically effective amount of a compound as defined in claim 1 or 4 and a pharmaceutically acceptable carrier or diluent.

6. A method of treating or preventing fungal infection in a mammal comprising administering to said mammal a therapeutically effective amount of a compound having the structure of Formula I 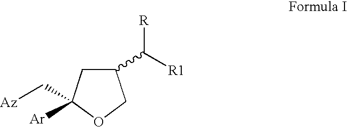
and its pharmaceutically acceptable salts, enantiomers, diastereomers, N-oxides, prodrugs, metabolites, polymorphs, or pharmaceutically acceptable solvates,
wherein
Az is a five to seven membered heterocyclic ring having one to four heteroatoms selected from N, S, or O; the preferred heterocyclic ring is 1,2,4-triazol-1-yl;
Ar is a five to seven membered heterocyclic ring containing one to four heteroatoms selected from the group consisting of oxygen nitrogen and sulphur; phenyl or a substituted phenyl group having one to three substituents independently selected from halogen (e.g. chlorine, fluorine, bromine or iodine), nitro, cyano, lower(C₁-C₄) alkyl, lower (C₁-C₄) alkoxy or a perhalo lower (C₁-C₄) alkyl, perhalo lower(C₁-C₄) alkoxy; the preferred heterocyclic rings are thienyl and pyridyl;
R is H or methyl;
R₁ is selected from the group consisting of 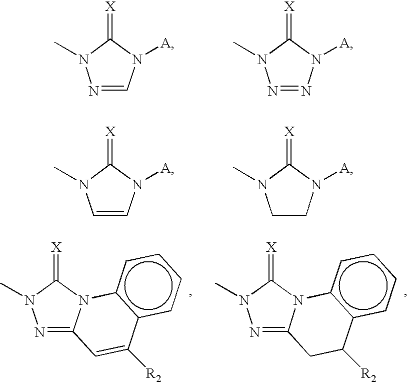
wherein
X is selected from the group consisting of CH₂, O, S and SO₂;
R₂ is hydrogen or lower(C₁-C₄) alkyl;
A is hydrogen, lower(C₁-C₄) alkyl, phenyl or phenyl substituted by one or more of groups independently selected from halogen (e.g. chlorine, fluorine, bromine or iodine atoms ), nitro, cyano, hydroxy, lower(C₁-C₄) alkyl, lower (C₁-C₄) alkoxy or perhalo lower (C₁-C₄) alkyl, perhalo lower (C₁-C₄)alkoxy ; substituted or unsubstituted five or six membered heterocyclyl ring systems containing one to four hetero atoms chosen from N, O and S, said heterocyclyl substituents being (C₁-C₈) alkanoyl, lower (C₁-C₄) alkyl, lower (C₁-C₄) alkoxy carbonyl, N,N-di(lower alkyl) (C₁-C₄) amino carbonyl, aminothiocarbonyl, N-lower(C₁-C₄) alkyl aminothiocarbonyl, N,N-di(lower alkyl) (C₁-C₄) aminothiocarbonyl, lower (C₁-C₄) alkyl sulfonyl, phenyl substituted lower (C₁-C₄) alkyl sulfonyl, N-lower (C₁-C₄) alkylamino, N,N-di(lower alkyl) (C₁-C₄) amino, 1,3-imidazol-1-yl, 2-loweralkyl(C₁-C₄) sulfenyl-1,3-imidazol-1-yl, pyridinyl, thaizolyl, 1,2,4 triazol-4-yl or phenyl or phenyl substituted by one or more of groups independently selected from halogen (chlorine, fluorine, bromine or iodine), perhalo lower(C₁-C₄) alkyl, (C₂-C₈) alkanoyl, lower(C₁-C₄) alkyl, lower(C₁-C₄) alkyl substituted by one or more hydroxy group, lower(C₁-C₄) alkoxy, nitro, cyano, hydroxy, 1,2,4-triazolyl, 1,3-imidazolyl, 1,2,3,4-tetrazolyl.

7. A method of treating or preventing a fungal infection in a mammal comprising the step of administering to said mammal a therapeutically effective amount of the pharmaceutical composition according to claim 5.

8. A process for preparing a compound of the Formula I, its pharmaceutically acceptable salts, enantiomers, diastereomers, N-oxides, prodrugs, metabolites, polymorphs, or pharmaceutically acceptable solvates
wherein
Az is a five to seven membered heterocyclic ring having one to four heteroatoms selected from N, S, or O; the preferred heterocyclic ring is 1,2,4-triazol-1-yl;
Ar is a five to seven membered heterocyclic ring containing one to four heteroatoms selected from the group consisting of oxygen nitrogen and sulphur; phenyl or a substituted phenyl group having one to three substituents independently selected from halogen (e.g. chlorine, fluorine, bromine or iodine), nitro, cyano, lower(C₁-C₄) alkyl, lower (C₁-C₄) alkoxy or a perhalo lower (C₁-C₄) alkyl, perhalo lower(C₁-C₄) alkoxy; the preferred heterocyclic rings are thienyl and pyridyl;
R is H or methyl;
R₁ is selected from the group consisting of 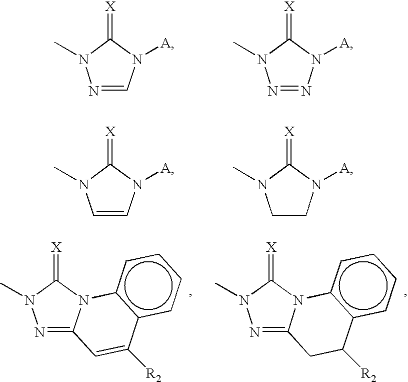
wherein
X is selected from the group consisting of CH₂, O, S and SO₂;
R₂ is hydrogen or lower(C₁-C₄) alkyl; A is hydrogen, lower(C₁-C₄) alkyl, phenyl or phenyl substituted by one or more of groups independently selected from halogen (e.g. chlorine, fluorine, bromine or iodine atoms), nitro, cyano, hydroxy, lower(C₁-C₄) alkyl, lower(C₁-C₄) alkoxy or perhalo lower (C₁-C₄) alkyl, perhalo lower(C₁-C₄)alkoxy; substituted or unsubstituted five or six membered heterocyclyl ring systems containing one to four hetero atoms chosen from N, O and S, said heterocyclyl substituents being (C₁-C₈) alkanoyl, lower (C₁-C₄) alkyl, lower (C₁-C₄) alkoxy carbonyl, N,N-di(lower alkyl) (C₁-C₄) amino carbonyl, aminothiocarbonyl, N-lower(C₁-C₄) alkyl aminothiocarbonyl, N,N-di(lower alkyl) (C₁-C₄) aminothiocarbonyl, lower (C₁-C₄) alkyl sulfonyl, phenyl substituted lower (C₁-C₄) alkyl sulfonyl, N-lower(C₁-C₄) alkylamino, N,N-di(lower alkyl) (C₁-C₄) amino, 1,3-imidazol-1-yl, 2-loweralkyl(C₁-C₄) sulfenyl-1,3-imidazol-1-yl, pyridinyl, thaizolyl, 1,2,4 triazol-4-yl or phenyl or phenyl substituted by one or more of groups independently selected from halogen (chlorine, fluorine, bromine or iodine), perhalo lower(C₁-C₄) alkyl, (C₂-C₈) alkanoyl, lower(C₁-C₄) alkyl, lower(C₁-C₄) alkyl substituted by one or more hydroxy group, lower(C₁-C₄) alkoxy, nitro, cyano, hydroxy, 1,2,4-triazolyl, 1,3-imidazolyl, 1,2,3,4-tetrazolyl;
which comprises reacting a compounds of Formula III with a compound of Formula IV as shown below, where all symbols are as defined above.

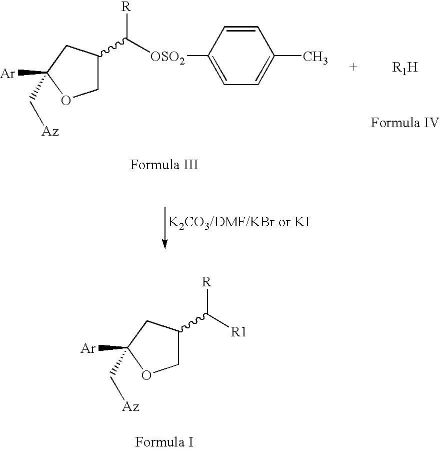

9. A process according to claim 8 for preparing a compound of the Formula II (Formula I, wherein Az is 1,2,4-triazol-1-lyl; R is H or CH₃; Ar is 2,4-dihalo substituted phenyl, Hal is Cl, F, Br or 1; and R₁ is 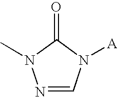
wherein A is the same as defined in claim 1, which comprises condensing 2,2,4-trisubstituted tetrahydrofuran of the Formula V with 4-substituted triazolone of Formula VI as shown below:

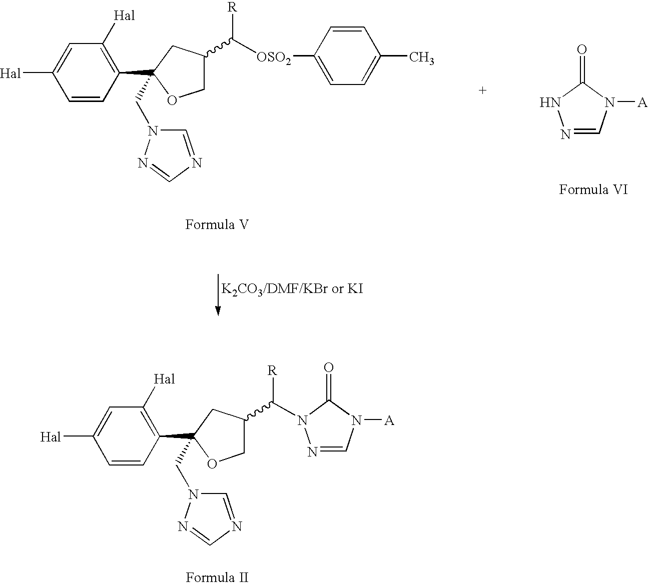

10. A process according to claim 9 for preparing a compound of Formula II, wherein A is represented by 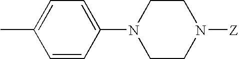
Z is a hydrogen, (C₁-C₈) alkanoyl, lower alkyl, (C₁-C₈) perhaloalkanoyl, or phenyl, phenyl substituted by one or more of groups independently selected from nitro, cyano, halogen (chlorine, fluorine bromine, iodine) perhalo lower(C₁-C₄) alkyl, perhalo lower(C₁-C₄) alkoxy; (C₂-C₈) alkanoyl, lower(C₁-C₄) alkyl, lower (C₁-C₄)alkyl substituted by one or more hydroxy group, lower(C₁-C₄) alkoxy, 1,3-imidazolyl, 1,2,4-triazolyl, 1,2,3,4-tetrazolyl, or OCH₂Y; wherein
Y is phenyl or phenyl substituted by one or more of groups independently selected from nitro, cyano, halo, perhalo lower alkyl, (C₂-C₈) alkanoyl lower alkyl, hydroxy, lower alkyl substituted by one or more hydroxy group, lower alkoxy, 1,3-imidazolyl, 1,2,4-triazolyl or 1,2,3,4-tetrazolyl.

11. The process of claim 8 wherein the reaction of compound of formula III and formula IV is carried out in a suitable organic solvent wherein the solvent is selected from the group consisting of dimethylformamide, dimethyl sulfoxide, toluene, isopropyl alcohol, tetrahydrofuran, ethylene glycol, dimethyl ether, and mixtures thereof.

12. The process of claim 8 wherein the reaction of compound of formula III and formula IV is carried out in the presence a suitable base.

13. The process of claim 12 wherein the base is selected from the group consisting of sodium hydride, potassium carbonate, cesium carbonate, and sodium carbonate.
